(19) United States
(12) Patent Application Publication
(10) Pub. No.: US 2009/0062852 A1
(43) Pub. Date: Mar. 5, 2009

(54) ANNULAR REPAIR DEVICE AND METHODS

(76) Inventor: James F. Marino, La Jolla, CA (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/199,642

(22) Filed: Aug. 27, 2008

Related U.S. Application Data
(60) Provisional application No. 60/966,930, filed on Aug. 29, 2007.

(51) Int. Cl. A61B 17/04; A61B 17/10

(52) U.S. Cl. 606/228; 606/232; 606/139

(57) ABSTRACT

Disclosed herein are devices, systems and methods to repair, bridge and protect defects in an annular ligament, such as an annulotomy site or other defect in the annulus fibrosis, wherein loops of suture are placed through the adjacent walls of the defect forming an “annular net” to prevent recurrent disc herniation. The devices and systems include a first and second suture and first and second suture delivery elements that are operatively associated with the first and second sutures and arranged to pass distal regions of the first and second sutures through an annular fibrosis adjacent the annular defect. The devices and systems also include a suture grasping member that grasps the distal regions of the sutures and retracts to pull the distal regions of the first and second sutures through the annular defect.

### REFERENCE TO PRIORITY DOCUMENT

This application claims the benefit of priority of U.S. Provisional Patent Application Ser. No. 60/966,930, entitled “Annular Repair Device and Methods”, filed Aug. 29, 2007. Priority of the aforementioned filing date is hereby claimed and the entire disclosure of which is hereby incorporated by reference.

### BACKGROUND

Intervertebral discs are soft structures that lie between adjacent vertebrae in the spine. The discs form a cartilaginous joint allowing slight movement of the vertebrae and act like ligaments to hold the vertebrae together. Each disc includes the inner nucleus pulposus and the outer annulus fibrosus. The nucleus pulposus is a soft, gel-like inner substance of the discs that act like shock absorbers and keep the vertebrae separated.

The annulus fibrosus is the surrounding tough, outer ring of the disc. If the annulus tears, such as due to an injury, the nucleus pulposus can begin to extrude through the tear in the annulus. This is called herniation of the disc. Although the nucleus pulposus is considered generally soft and gel-like, in a majority of clinical herniations the nucleus pulposus presents in a degenerative state as a fibrous collagenized mass. Depending upon the location and type of herniation a variety of symptoms can result including pain, spasm, weakness, and numbness due to the nucleus pulposus pushing out and compressing the nerve root.

A common treatment for a herniated disc is surgery, such as spinal discectomy. During a discectomy a surgeon will remove the extruding disc fragment that is compressing the nerve root and causing the painful symptoms. Following such a procedure, a defect will remain in the annulus. Annular defects are susceptible to recurrent disc herniation and the need for re-operation.

### SUMMARY

There is a growing need for improved methods and devices to repair annular defects and prevent recurrent disc herniation after surgical discectomy procedures.

Disclosed in the present application are devices and systems for repairing a defect in an annular ligament. The devices and systems include a first and second suture each having a proximal region and a distal region. First and second suture delivery elements are also included that are operatively associated with the first and second sutures and arranged to pass distal regions of the first and second sutures through an annular fibrosis adjacent the annular defect. The devices and systems also include a suture grasping member that grasps the distal regions of the sutures and retracts to pull the distal regions of the first and second sutures through the annular defect.

Also disclosed in the present application are methods of repairing a defect in an annular ligament by providing a first suture and a second suture each having a proximal region and a distal region; threading the distal regions of the first and second sutures through an annulus fibrosis adjacent to an annular defect; retrieving the distal regions of the first and second sutures threaded through the annulus fibrosis back through the annular defect; securing the distal region of the first suture to a portion of the first suture to form a first loop that extends through the annulus fibrosis and through the annular defect; securing the distal region of the second suture to a portion of the second suture to form a second loop that extends through the annulus fibrosis and through the annular defect; and attaching the first suture to the second suture to form a net that spans the annular defect.

Other features and advantages will be apparent from the following description of various embodiments, which illustrate, by way of example, the principles of the disclosed devices and methods.

### BRIEF DESCRIPTION OF THE DRAWINGS

FIG. 1A shows a perspective view of one embodiment of the annular repair device.

FIG. 1B shows a cross-section of the device of FIG. 1A taken along line 1B.

FIG. 1C shows a detailed view of the distal end of the device of FIG. 1A taken along circle 1C.

FIG. 2 shows a perspective view of one embodiment of the needle-suture assembly of the device of FIG. 1.

FIG. 3 shows a perspective view of one embodiment of the suture snare assembly of the device of FIG. 1.

FIGS. 4A-4G illustrate an exemplary method of treating an annular defect using an embodiment of the device disclosed herein.

FIGS. 5A-5C illustrate an exemplary method of locking the suture into the tissue surrounding the annular defect.

FIGS. 6A-6C illustrate exemplary suture configurations to treat an annular defect using an embodiment of the device disclosed herein.

### DETAILED DESCRIPTION

Disclosed herein are devices and methods to repair, bridge and protect defects in an annular ligament, such as an annulotomy site or other defect in the annulus fibrosis, wherein loops of suture are placed through the adjacent walls of the defect forming an “annular net” to prevent recurrent disc herniation. Although the devices and methods are described herein as they relate to spinal surgery and the treatment of annular defects, it is understood that the technology can be used for other surgical procedures, for example minimally-invasive vascular repair following various interventional procedures (e.g. arteriography and stent insertion).

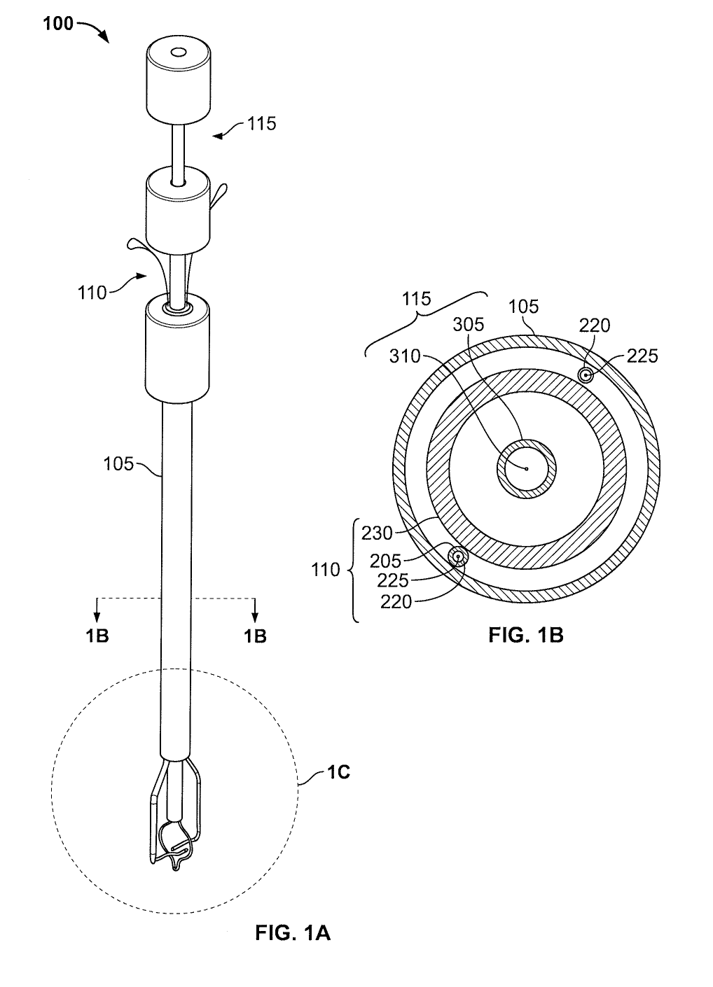

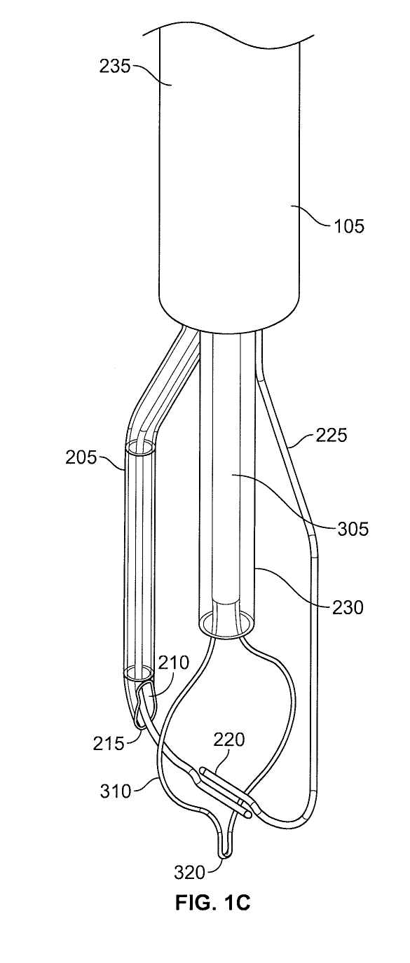

FIG. 1A shows a perspective view of one embodiment of an annular repair device 100. The device 100 generally includes a tubular repair isolation sleeve 105, a suture delivery element or needle-suture assembly 110 and a suture snare assembly 115. The repair isolation sleeve 105 is a sheath, such as a thin-walled tubular sheath, through which the needle-suture assembly 110 and suture snare assembly 115 are slideably disposed. The repair isolation sleeve 105 assists in deploying the needle-suture assembly 110 and suture snare assembly 115 to the target location, such as a defect in an annulus of the spine, to be described in more detail below. As best shown in FIG. 1B, the repair isolation sleeve 105 surrounds the needle-suture assembly 110 which surrounds the suture snare assembly 115.

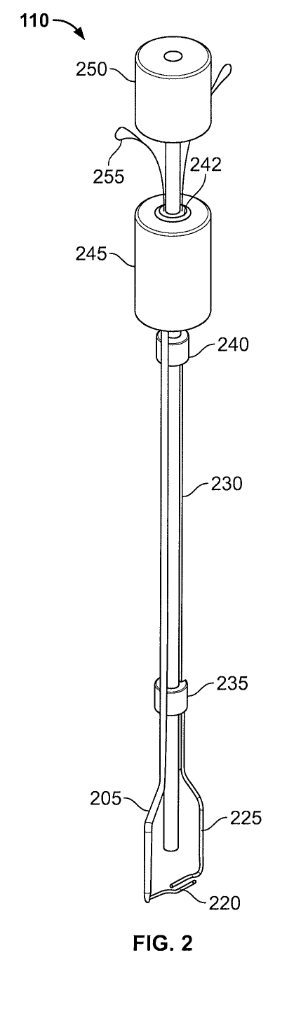

FIG. 2 shows a perspective view of one embodiment of the needle-suture assembly 110 of the device 100. The needle-suture assembly 110 includes a plurality of needles or needle pairs 205, sutures 220 and a tubular central element 230. The needle-suture assembly 110 of FIGS. 1A, 1B, 1C and 2 show only one needle 205. It should be appreciated that pairs of needles, for example 1, 2 or 3 or 4 or more pairs of needles 205 are considered. It also should be appreciated that for every needle 205 employed by the device 100, an associated suture 220 can be employed.

In an embodiment, each needle 205 has a non-linear geometry such that the distal region extends a radial distance from the proximal axis of otherwise linear or near-linear needle shaft. In one embodiment, the needles have roughly the contour of a “swan's neck” (best shown in FIG. 1C). That is, distal regions of the needles flare outward with respect to a longitudinal central axis of the sleeve 105. The needles 205 can be non-coring and/or can be stainless steel or other medical grade material. In an embodiment, the needles 205 can be a shape-memory material such as nitinol.

At their distal end, the needles 205 can have a sharp, beveled tip 215. The beveled tip 215 of the needles 205 can be biased toward the midline of the device 100. The needles 205 can have a cross-sectional geometry, such as a triangular or other angular geometry, that provides rotational stability when engaged or wedged together with, for example, the central element 230.

The needles 205 can have a central, smooth-walled bore 210 through which suture 220 (loops or free ends) can be fed in order to thread the suture through the wall of the annulus fibrosis. In an embodiment, the suture 220 can be fed through the bore 210 of the needle 205 by hydraulic flow. A fluid source connected at or near the proximal end of the needles 205 can be used to infuse fluid through the bore 210 of the needles 205 carrying with it the suture 220. This infusion of fluid through the needles 205 delivers the suture 220 out through the distal tip of the needles 205. The fluid source can include, for example, a syringe filled with saline or the like. In another embodiment, the suture 220 can be looped such that a guide wire 225 can be inserted through the suture 220 and used to feed the suture 220 through the bore 210 of the needle 205 and out past the distal tip 215. The guide wires 225 can be made of a shape-memory material such as nitinol such that upon exiting the distal tip of the needles 205, the guide wires 225 veer toward the midline. Once near the midline, the suture 220 is more easily retrieved back up through the device such as by suction or using a retrieval device such as a suture snare. The remainder of the suture 220 can be closely apposed to either side of the needle 205 within the bore 210. For example, the suture 220 can be frangibly fixed to a recess within the bore 210 of the needle 205 or to the guide wire 225 such as with an adhesive like cyanoacrylate adhesive or a water soluble adhesive. Upon insertion of the needle 205 the suture 220 can be dissociated from the bore 210 such as by mechanical force or by dissolution of the adhesives such that irrigation with an appropriate solvent releases the suture 220 from the needle 205 for suture retrieval. The proximal ends 255 of the suture can be “free” or they can be looped, knotted or the like. The suture 220 can be, for example, braided suture or any other suture known in the art. Braided suture can be coated and/or impregnated with polytetrafluoroethylene (PTFE) (e.g. TEVDEK) to decrease friction and wicking as well as improve stiffness and strength of the suture.

Still with respect to FIG. 2, the needle-suture assembly 110 also includes a central element 230 slideably disposed inside the repair isolation sleeve 105. The distal end of the central element 230 is open and the proximal end of the central element 230 can be fixed to a grip 250 or can be attached to a vacuum source to assist in suction suture retrieval as described in more detail below. The central element 230 is generally tubular, but can have other geometries. Similarly, the diameter of the central element 230 can vary. In an embodiment, the central element 230 has a region near its distal end that has a reduced diameter compared to the more distal end. The central element 230 lies between opposing needle pairs along the midline or long axis of the device 100. The central element 230 is involved in the fixation of deployed needle pairs, to be described in more detail below.

The needle pairs can be mounted onto one or more needle actuators 240, 242. In addition, the needle pairs can be positioned on a needle mount 235 more distally. The needle mount 235 and needle actuators 240, 242 can be donut-shaped and surround the central element 230. The needle mount 235 and needle actuators 240, 242 also can have notches which engage the shafts of the needle pairs thereby fixing them in place within the device. It should be appreciated that the structural configuration of the needle actuators 240, 242 and the needle mount 235 can vary.

The needle actuator 242 can be attached and surrounded by a needle actuator grip 245. During actuation of the device, the operator holding the grip 245 can advance the needle pairs distally with respect to the central element 230 and the repair isolation sleeve 105. The distal edge of the grip 245 can slide over needle actuator 240. Needle actuator 242 cannot slide over needle actuator 240. Thus, the needle actuator 240 acts as a distal stop and prevents the proximal needle actuator 242 from being advanced beyond the position of needle actuator 240.

As mentioned above, the proximal end of the central element 230 is involved in fixation of the deployed needle pairs. During actuation of the device, the operator can advance the central element 230 distally with respect to the needle pairs 205 and also the repair isolation sleeve 105. Advancing the central element 230 in the distal direction a particular distance can wedge the deployed needles 205 up against the inner wall of the repair isolation sleeve 105. This fixes the deployed needles 205 and stabilizes any lateral flexibility of the needles 205 such that they can, for example, pierce the annular tissue. Because the central element 230 can have a region of decreased diameter near its distal end, further advancing the central element 230 in the distal direction can release the deployed needles 205 from their fixed position against the inner wall of the sleeve 105.

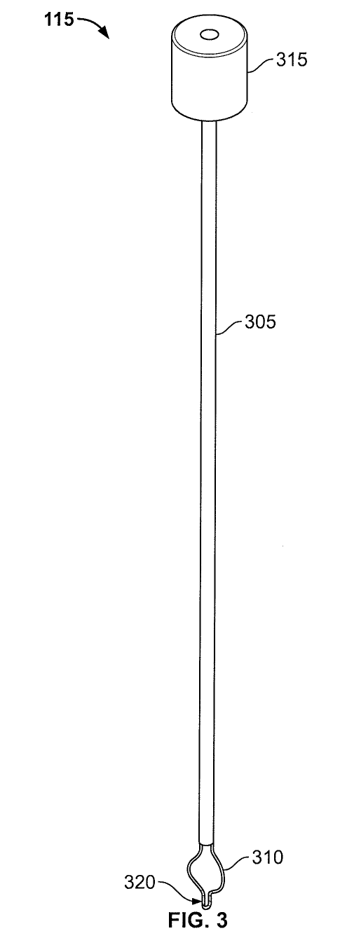

After the needle pairs pierce the target tissue, suture (including suture loops, free ends, etc.) can be fed through the bore of the needles and thread through the wall of the annulus fibrosis. The method of suture delivery can vary. For example, suture can be delivered by hydraulic flow through the needle bore or using guide wires to mechanically push the suture through the bore. The suture can also be retrieved back out through the defect by a variety of methods. In an embodiment, threaded suture is retrieved by a suture snare assembly. FIG. 3 shows a perspective view of an embodiment of a suture snare assembly 115. The suture snare assembly 115 can be positioned inside the central element 230, which is positioned inside the repair isolation sleeve 105. The suture snare assembly 115 can include a suture grasping member such as a suture snare 310 fixed within a tubular suture recovery sleeve 305 and can have a grip 315 near its proximal end. The suture recovery sleeve 305 can be slideably disposed inside the central element 230 of the needle-suture assembly 110. The suture snare 310 acts as a capture device to retrieve suture 220 delivered by the needle pairs 205. The suture snare 310 can be a loop of medical grade material, including a shape-memory material like nitinol. The snare 310 can have a region 320 in the internal loop that has a reduced dimension. This reduced dimension region 320 assists in capturing and securing the suture within the snare 310 upon retraction.

In another embodiment, the device does not include a suture snare assembly. In this embodiment, retrieval of suture is performed using suction, for example, through the central element. In another embodiment, the device includes a suture delivery/retrieval relay mechanism employing both hydraulic flow and suction. In this embodiment, the relay mechanism delivers suture through the bores 210 of the needle pair 205 using hydraulic flow and then retrieves the suture 220 through the central element 230 by suction. A fluid source connected at or near the proximal end of the needle pairs 205 can be used to infuse fluid through the bores 210 of the needle pair 205 carrying with it the suture 220. This infusion of fluid through the needle pair 205 delivers the suture 220 out the distal tip of the needle pair 205. The fluid source can include, for example, a syringe filled with saline or the like. Once the suture 220 is deployed into the annular defect, it is retrieved, for example, by suction up through the central element 230. It should be appreciated that the embodiment can incorporate a trigger-type relay mechanism for the user to easily deploy and retrieve the suture. In this embodiment, the user upon actuating, for example, a trigger on the device would initiate a two-step relay mechanism in which a fluid flow step delivering the suture through the bore of the needle into the annular defect is immediately followed by a suction step in which suture is retrieved from the annular defect up through the central element. The two-step relay mechanism would feel to the user to be nearly instantaneous and as a result of one step or one trigger pull. Thus, upon actuating the trigger of the device the needles are then withdrawn leaving suture loops in place at the tissue surrounding the annular defect.

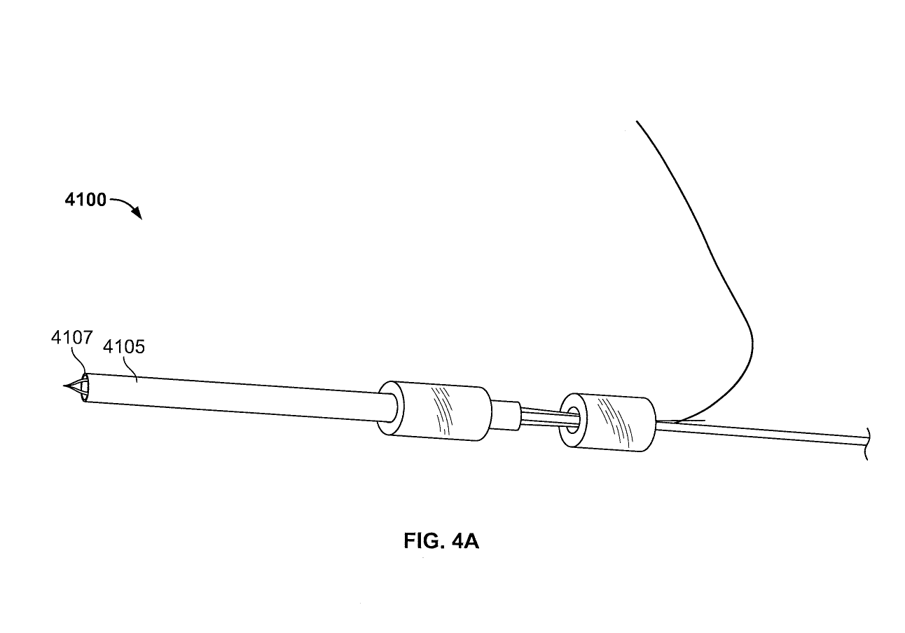

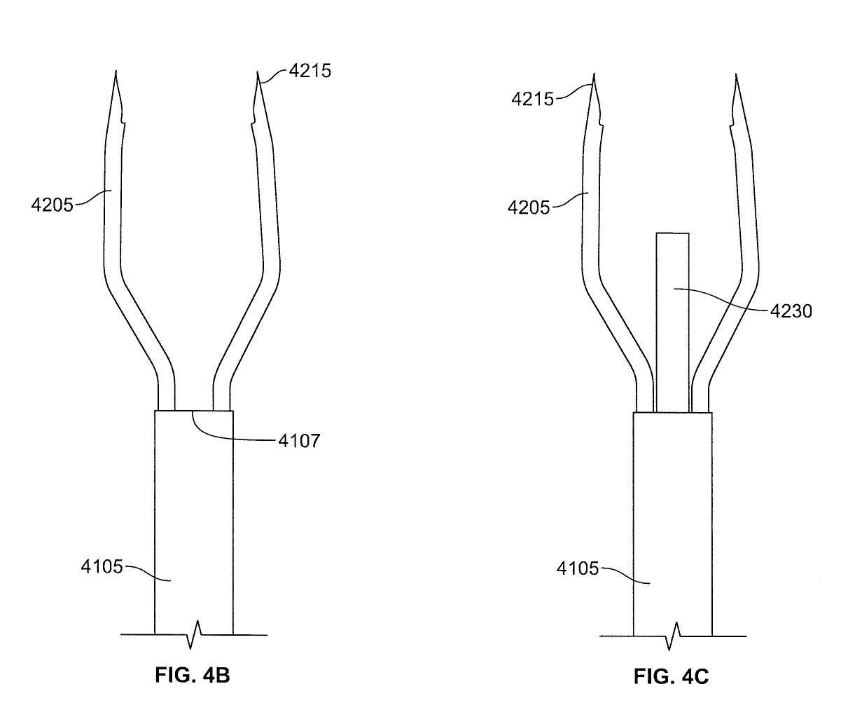

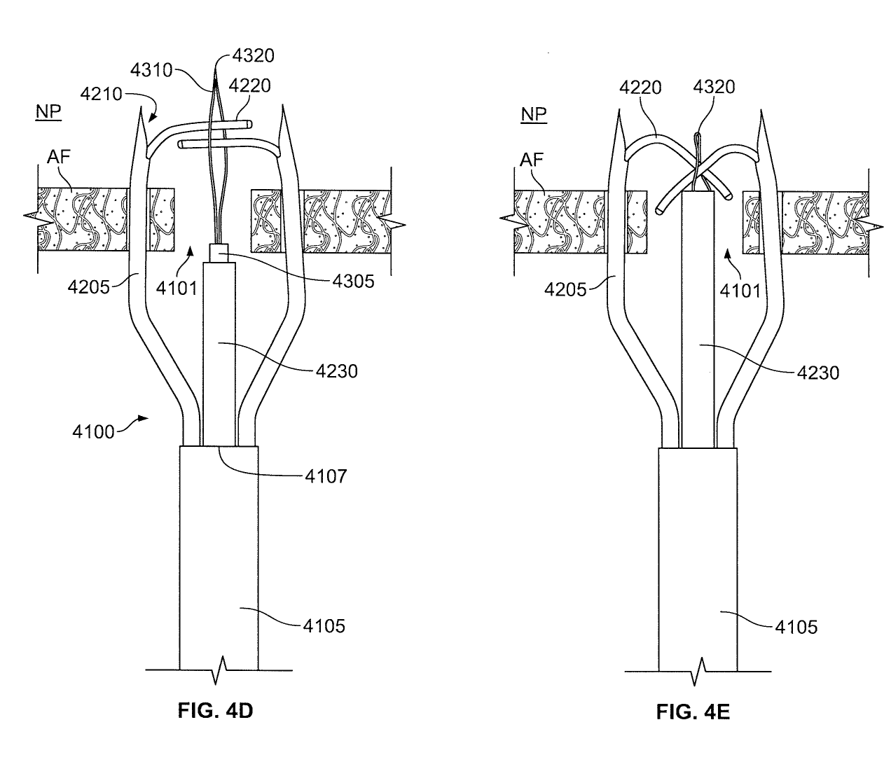

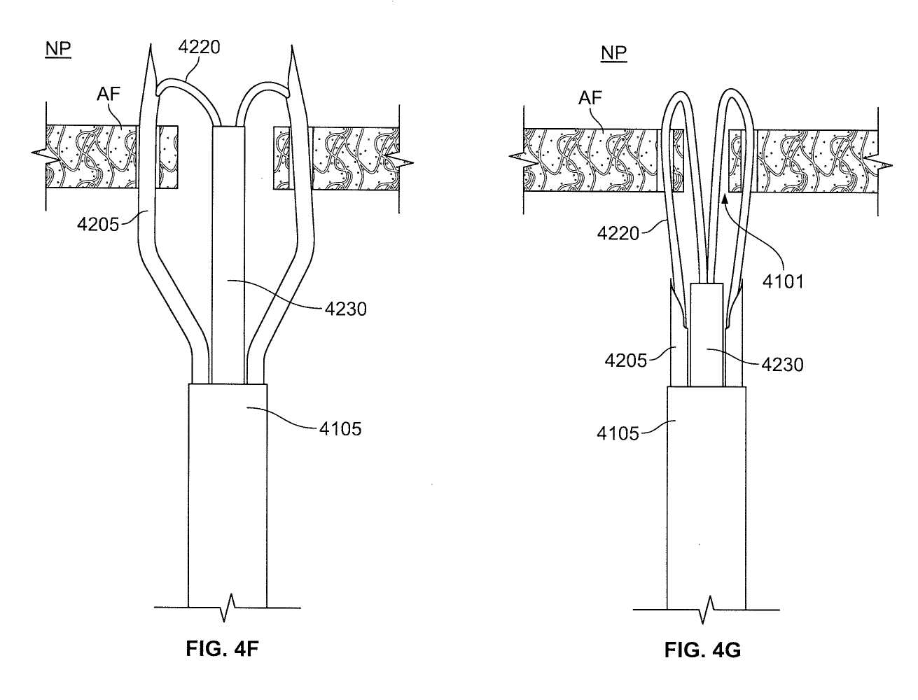

FIGS. 4A-4G illustrate an exemplary method of treating an annular defect using an embodiment of the device disclosed herein. As shown in FIG. 4A, the needle pair is initially completely contained within the repair isolation sleeve 4105 of the device 4100. This is the “sheathed position” used just prior to needle deployment. FIG. 4B shows the needle pair 4205 advanced beyond the distal edge 4107 of the repair isolation sleeve 4105, resulting in the needles 4205 taking on a splayed position. This “relaxation” can be due to the configuration of the needle pair 4205 within the repair isolation sleeve 4105. In an embodiment, the needles 4205 can have a shape, such as a stepped shape, that when retracted in the repair isolation sleeve 4105 the needle pair 4205 crosses the midline of the sleeve 4105. Upon advancing the needle pair 4205 beyond the distal end of the sleeve 4105, they gradually uncross and splay into a position appropriate for piercing the tissue at the perimeter of the annular defect. In another embodiment, the needle pair 4205 is made of nitinol memory metal and upon deployment from the sleeve 4105, the metal “relaxes” into the splayed position.

FIG. 4C shows the central element 4230 advanced distally a short distance. Advancement of the central element 4230 further splays the needle pair 4205 stabilizing them by, for example, wedge-shaped mating geometry or opposing surfaces that pushes the needle pair 4205 against the inner wall of the repair isolation sleeve 4105.

FIG. 4D illustrates an embodiment of the device in which a suture snare 4310 is deployed to capture and retrieve the suture 4220. The suture snare 4310 is deployed by advancing the suture recovery sleeve 4305 in the distal direction. The device 4100 is then advanced through the annular defect 4101 in the annulus fibrosus AF. The distal tips 4215 of the needle pair 4205 pierce the tissue at the perimeter of the defect 4101 until the needle pair 4205 emerges several millimeters, for example, within the void created by the resection of the herniated nucleus pulposus NP. This is estimated to be between 5 mm and 10 mm although the distance can vary.

Once the needles 4205 have pierced the tissue surrounding the defect 4101, the suture 4220 can be deployed such that it threads through the wall of the annulus fibrosis. The suture 4220 can be deployed, for example, using hydraulic flow as described above. Alternatively, the suture 4220 can be deployed using a guide wire to advance the suture 4220 through the needle bore and out the distal tip of the needle 4205. The suture 4220 approaches the midline of the device such that the suture 4220 can be captured by the snare 4310 for retrieval. Alternatively, the device does not include a snare 4310 for retrieving the suture and instead employs suction through the central element 4320 to retrieve the suture.

The snare 4310 can pull and separate the suture 4220 from the bore 4210 of the needle 4205. As shown in FIGS. 4E and 4F, the suture recovery sleeve 4305 and the suture snare 4310 are retracted to capture the suture 4220. The internal loop of the suture snare 4310 can have region 4320 that is of a reduced dimension. The region 4320 assists in capturing and securing the suture 4220. Retracting the snare 4310 pulls the suture 4220 out through the annular defect 4101 into the central element 4230. As the snare 4310 is pulled into the central element 4230 the walls of the snare 4310 can cross helping to further capture and secure the suture 4220. The central element 4230 and suture snare 4310 are withdrawn pulling the suture 4220 to the proximal region of the device. The needles are then withdrawn leaving suture loops in place at the tissue surrounding the annular defect 4101 (shown in FIG. 4G).

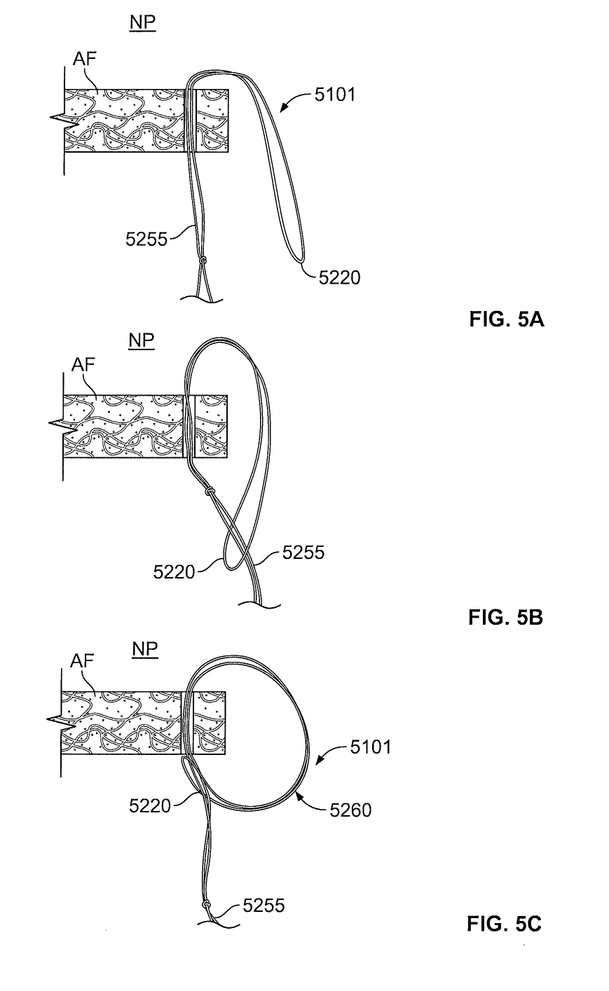

The sutures delivered to the tissue surrounding an annular defect can be locked into place using a variety of techniques. FIGS. 5A-5C illustrate one embodiment. In this embodiment, the suture is looped near its distal end 5220 and the proximal “free” ends 5255 are associated together. The associated proximal ends 5255 can be easily fed through or wrapped around the suture loop 5220 that is retrieved through the annular defect 5101 as described above such as by a snare or by suction. To assist in the mating of the looped ends 5220 with their respective proximal ends 5255, each suture can vary in color. For example, if two pairs of needles are employed, four varied-colored sutures can be used. The proximal ends 5255 can be placed through or around their respective color-matched suture loops 5220 in a slip collar-type formation as shown in FIG. 5B. The proximal ends 5255 can be gently pulled until the slack is removed from the suture material. This results in four collars 5260 of suture at opposing edges of the annular defect. One opposing edge with a collar 5260 is shown in FIG. 5C.

Once the collars of suture are in place at the edges or perimeter of the defect, a suture net or bridge that spans the defect can be formed. In one embodiment, distal regions of the suture are fixed together, such as the looped distal ends 5220 described above. In another embodiment, the proximal regions of the suture are fixed together, such as the free proximal ends 5255 described above. Whether distal or proximal regions of the suture are fixed together, the end result is that a net or bridge is formed across the defect.

The suture can be fixed, for example, by bonding with agents like cyanoacrylic adhesive, by thermal association to melt suture regions together, a loop of suture knotted around the suture regions, or a suture clamp or clip. In an embodiment, the suture regions can be placed through a small oval ring of titanium at the end of a suture tensioning and titanium ring crimping device. While maintaining slight and equal tension on the suture regions, the suture tensioning and titanium ring crimping device can be advanced within the repair isolation sleeve into the target treatment area. The crimping device's grip handle can be squeezed crimping the titanium oval ring thus securing the suture regions. Such devices can also cut the suture strands just proximal to the crimped ring. The suture loops, knots and clamps etc. can be advanced within the central element into the target treatment area using, for example, hydraulic flow.

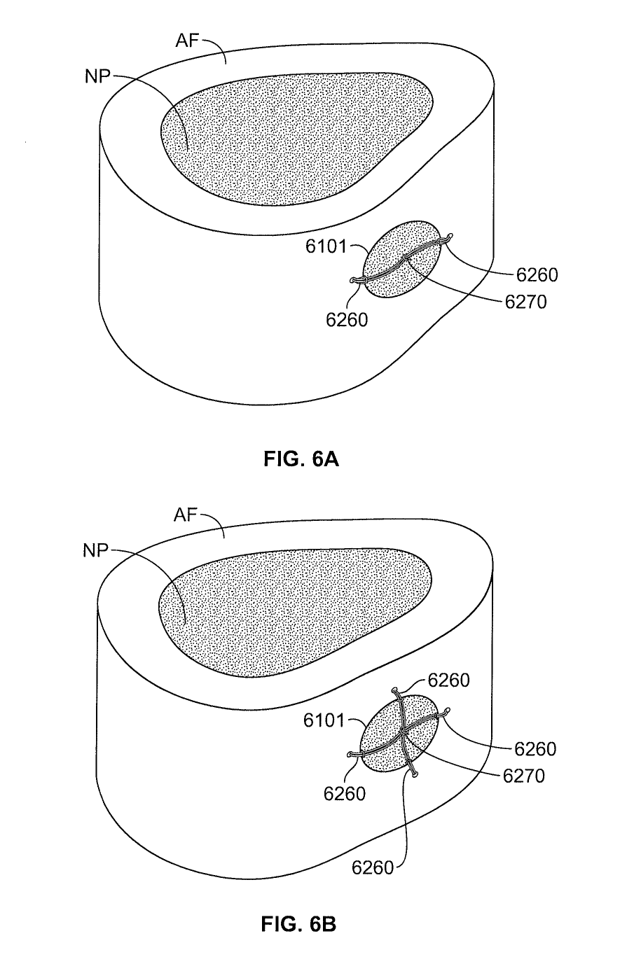

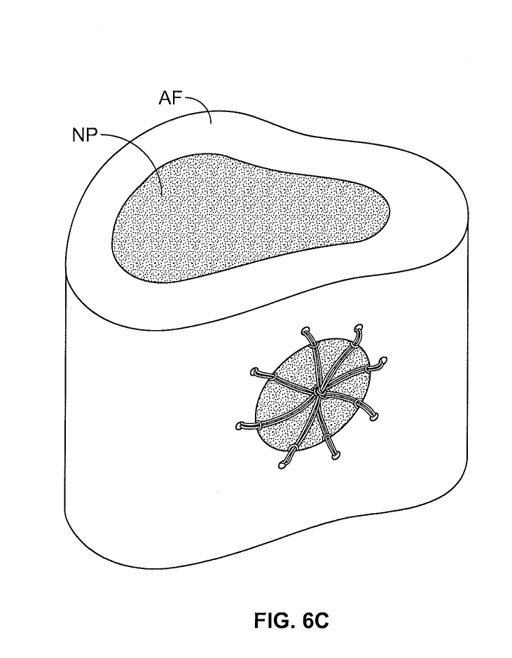

FIGS. 6A-6C illustrate exemplary resultant suture net configurations spanning the annular defect. The exemplary methods described above employ a device having one pair of needles to deliver and create two suture loops through the tissue. In such an embodiment, the resulting suture configuration would resemble what is shown in FIG. 6A in which two suture loops 6260 are delivered to tissue surrounding the annular defect 6102. The suture loops 6260 can be delivered, for example, using a technique as described above. The suture spans the defect 6101 and can be fixed as described above such as with a crimping ring 6270. FIG. 6B illustrates an embodiment in which two pairs of needles are used to deliver four suture loops 6260. FIG. 6C illustrates an embodiment in which four needles are used to deliver eight suture loops. The resulting suture configuration acts essentially like a bridge or net across the defect thereby creating an obstruction. The net of suture impedes material from leaving the intradiscal space via the annular defect and thereby prevents or mitigates recurrent disc herniation and the need for further surgical procedures.

Although the embodiments illustrated herein show passing the suture from the superficial surface of the annulus to the deep surface, it should be understood that suture can also be passed from the deep surface to the superficial surface of the annulus.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Only a few examples and implementations are disclosed. Variations, modifications and enhancements to the described examples and implementations and other implementations may be made based on what is disclosed.

What is claimed is:

1. A device for repairing a defect in an annular ligament, comprising:
a first suture and a second suture each having a proximal region and a distal region;
first and second suture delivery elements operatively associated with the first and second sutures and arranged to pass distal regions of the first and second sutures through an annular fibrosis adjacent the annular defect; and
a suture grasping member that grasps the distal regions of the sutures, wherein the suture grasping member retracts to pull the distal regions of the first and second sutures through the annular defect.

2. A device as in claim 1, further comprising a tubular isolation sleeve that surrounds the first and second suture delivery elements and the first and second sutures and wherein the first and second suture delivery elements and the first and second sutures are slideably positioned in the tubular isolation sleeve.

3. A device as in claim 2, further comprising a tubular central element co-axially positioned within the tubular isolation sleeve with the first and second suture delivery elements interposed between the tubular central element and the tubular isolation sleeve, wherein the tubular central element advances with respect to the first and second suture delivery elements and the tubular isolation sleeve thereby pushing the first and second suture delivery elements against an inner wall of the tubular isolation sleeve and stabilizing lateral flexibility of the first and second suture delivery elements.

4. A device as in claim 3, further comprising a suture recovery sleeve co-axially positioned inside the tubular central element and the tubular isolation sleeve, wherein the suture grasping member is contained within the suture recovery sleeve such that the suture grasping member pulls the distal regions of the first and second sutures through the annular defect into the suture recovery sleeve.

5. A device as in claim 1, wherein the suture grasping member is a loop comprising a region of reduced dimension.

6. A device as in claim 1, wherein the suture grasping member comprises a suction source that sucks the distal ends of the sutures into a suture recovery sleeve.

7. A device as in claim 1, wherein the first and second sutures are slideably disposed within the first and second suture delivery elements, respectively.

8. A device as in claim 7, further comprising a guidewire to feed the first and second sutures through the first and second suture delivery elements.

9. A device as in claim 1, wherein distal regions of the first and second suture delivery elements flare outward with respect to a central longitudinal axis of the device.

10. A device as in claim 9, wherein distal tips of the first and second suture delivery elements are biased toward the central longitudinal axis.

11. A device as in claim 1, wherein the first and second sutures are different colors.

12. A device as in claim 1, wherein the first and second sutures are frangibly associated with the first and second suture delivery elements.

13. A device as in claim 12, further comprising an adhesive that frangibly associates the first and second sutures to the first and second suture delivery elements.

14. A device as in claim 13, wherein the adhesive comprises cyanoacrylate.

15. A device as in claim 13, wherein the adhesive comprises a water-soluble adhesive.

16. A device as in claim 13, wherein irrigation through the suture delivery element dissolves the adhesive and releases the first and second sutures from the first and second suture delivery elements.

17. A device as in claim 13, wherein the first and second sutures are mechanically released from the first and second suture delivery elements.

18. A method of repairing a defect in an annular ligament, comprising:
providing a first suture and a second suture each having a proximal region and a distal region;
threading the distal regions of the first and second sutures through an annulus fibrosis adjacent to an annular defect;
retrieving the distal regions of the first and second sutures threaded through the annulus fibrosis back through the annular defect;
securing the distal region of the first suture to a portion of the first suture to form a first loop that extends through the annulus fibrosis and through the annular defect;
securing the distal region of the second suture to a portion of the second suture to form a second loop that extends through the annulus fibrosis and through the annular defect; and
attaching the first suture to the second suture to form a net that spans the annular defect.

19. A method as in claim 18, wherein securing the distal region of the first suture to the portion of the first suture comprises using a slip collar.

20. A method as in claim 18, wherein threading the distal regions of the first and second sutures through the annulus fibrosis comprises using hydraulic flow.

21. A method as in claim 18, wherein providing a first and second suture comprises providing first and second suture delivery elements wherein the first and second sutures are frangibly associated with the first and second suture delivery elements.

22. A method as in claim 21, wherein an adhesive is employed to frangibly associate the first and second sutures to the first and second suture delivery elements.

23. A method as in claim 21, further comprising mechanically releasing the first and second sutures from the first and second suture delivery elements.

24. A method as in claim 22, wherein the adhesive comprises cyanoacrylate.

25. A method as in claim 22, wherein the adhesive comprises a water-soluble adhesive.

26. A method as in claim 22, further comprising dissolving the adhesive releasing the first and second sutures from the first and second delivery elements prior to retrieving the distal regions of the first and second sutures threaded through the annulus fibrosis.

27. A method as in claim 18, further comprising using a guidewire to guide the first and second sutures to assist in threading of the distal regions of the first and second sutures through the annulus fibrosis.

28. A method as in claim 18, wherein retrieving the distal regions of the first and second sutures threaded through the annulus fibrosis back through the annular defect comprises grasping the distal regions with a loop comprising a region of reduced dimension.

29. A method as in claim 18, wherein retrieving the distal regions of the first and second sutures threaded through the annulus fibrosis back through the annular defect comprises using suction.

30. A method as in claim 18, wherein providing a first suture and a second suture comprises providing the first suture of a first color and providing the second suture of a second color different from the first color.

31. A method as in claim 18, wherein attaching the first suture to the second suture comprises using an adhesive.

32. A method as in claim 18, wherein attaching the first suture to the second suture comprises using a device to attach a portion of the first suture to a portion of the second suture, the device selected from the group consisting of a clip, a clamp, a tensioning ring, and a crimping ring.

33. A method as in claim 18, wherein attaching the first suture to the second suture comprises providing a third suture loop to attach a portion of the first suture to a portion of the second suture.

34. A method as in claim 18, wherein attaching the first suture to the second suture comprises melting a portion of the first suture to a portion of the second suture.

35. A method as in claim 18, further comprising:
providing additional sutures each having a proximal region and a distal region;
threading the distal regions of additional sutures through the annulus fibrosis adjacent to an annular defect;
retrieving the distal regions of the additional sutures back through the annular defect;
attaching the distal region of each additional suture to a portion of itself to form additional loops that extend through the annulus fibrosis and through the annular defect; and
attaching the additional sutures to one another and to the first and second sutures to form a net that spans the annular defect.
